(19) United States
(12) Patent Application Publication
(10) Pub. No.: US 2007/0281932 A1
(43) Pub. Date: Dec. 6, 2007

(54) Method of preparing 4-halogenated quinoline intermediates

(75) Inventors: Caroline Bernier, Otterburn Park (CA); Chia-Cheng Shaw, St-Laurent (CA)

Correspondence Address: FITZPATRICK CELLA (WYETH), 30 ROCKEFELLER PLAZA, NEW YORK, NY, 10112-3800, US

(73) Assignee: WYETH, Madison, NJ (US)

(21) Appl. No.: 11/802,430

(22) Filed: May 23, 2007

Related U.S. Application Data
(60) Provisional application No. 60/802,759, filed on May 23, 2006.

(51) Int. Cl. A61K 31/5377; A61K 31/496; A61K 31/47; C07D 413/02; C07D 403/02

(52) U.S. Cl. 514/232.8; 514/253.06; 514/311; 514/314; 544/128; 544/363; 546/176

(57) ABSTRACT

This invention is directed to methods of preparing compounds of formula (I): 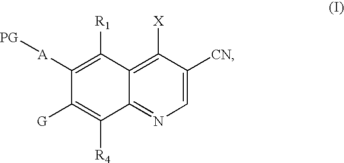
comprising the step of reacting a compound of formula (II): 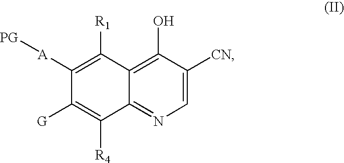
with a reagent of formula POX₃ and silica gel at a temperature greater than about 75° C., and wherein substitutions at X, PG, A, G, R₁ and R₄ are set forth in the specification.

### CROSS-REFERENCE TO RELATED APPLICATIONS

This application claims the benefit of U.S. Provisional Application No. 60/802,759, filed May 23, 2006, which is incorporated herein by reference.

### BACKGROUND OF THE INVENTION

1. Field of the Invention

This invention is directed to methods of preparing 4-halogenated quinoline compounds as intermediates in the manufacture of biologically active compounds, for example receptor tyrosine kinase inhibitors.

2. Related Background Art

Protein tyrosine kinases (PTKs) are critical in regulating cell growth and differentiation. One general class of PTK is the receptor tyrosine kinase (RTK). Once activated, usually through the binding of a ligand, an RTK initiates signaling for various activities, such as cell growth and replication.

The RTKs comprise one of the larger families of PTKs and have diverse biological activity. At present, at least nineteen (19) distinct subfamilies of RTKs have been identified. One such subfamily is the “HER” family of RTKs, which includes epidermal growth factor receptor (EGFR), ErbB2 (HER2), ErbB3 (HER3) and ErbB4 (HER4).

Under certain conditions, as a result of either mutation or over expression, studies have shown that these RTKs can become deregulated; the result of which is uncontrolled cell proliferation which can lead to tumor growth and cancer (Wilks, A. F., Adv. Cancer Res., 60, 43 (1993) and Parsons, J. T.; Parsons, S. J., Important Advances in Oncology, DeVita, V. T. Ed., J. B. Lippincott Co., Phila., 3 (1993)). For example, over expression of the receptor kinase product of the ErbB2 oncogene has been associated with human breast and ovarian cancers (Slamon, D. J. et al., Science, 244, 707 (1989) and Science, 235, 177 (1987)).

In addition, deregulation of EGFR kinase has been associated with epidermoid tumors (Reiss, M., et al., Cancer Res., 51, 6254 (1991)), breast tumors (Macias, A. et al., Anticancer Res., 7, 459 (1987)), and tumors involving other major organs (Gullick, W. J., Brit. Med. Bull., 47, 87 (1991)).

These RTKs are known to also be involved in processes crucial to tumor progression, such as apoptosis, angiogenesis and metastasis.

Therefore, inhibitors of these RTKs have potential therapeutic value for the treatment of cancer and other diseases characterized by uncontrolled or abnormal cell growth. Accordingly, many recent studies have dealt with the development of specific RTK inhibitors as potential anti-cancer therapeutic agents (e.g., Traxler, P., Exp. Opin. Ther. Patents, 8, 1599 (1998) and Bridges, A. J., Emerging Drugs, 3, 279 (1998)).

Quinoline derivatives are known to be important intermediate compounds in the synthesis of RTK inhibitors. For example, in the following US patents, quinoline derivatives are disclosed and the compounds are stated to be involved in inhibiting PTK activity: U.S. Pat. No. 6,288,082 (Sep. 11, 2001) and U.S. Pat. No. 6,297,258 (Oct. 2, 2001).

In addition, various methods for the preparation of 4-halogenated quinoline intermediates are known in the art, but these methods contain serious limitations such as the generation of unwanted by-products. For example, the chlorination reaction used in preparing 4-chloroquinoline derivatives suffers from the generation of viscous tars and decomposition products that are difficult to clean, remove, and impede stirring on large scale preparation, which results in yields that vary widely, typically in the range from 30-50%, unless a large excess of the halogenating reagent is used, whereby yields may approach 60%.

Accordingly, there continues to be a need for novel methods of preparing 4-halogenated quinoline compounds used in the preparation of RTK inhibitors in high-yield and in a cost effective manner.

### SUMMARY OF THE INVENTION

This invention relates to methods of preparing 4-halogenated quinoline compounds as intermediates in the manufacture of biologically active compounds, such as RTK inhibitors.

Thus, the present invention is a method of preparing a compound of formula (I):

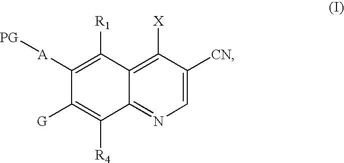

comprising the step of reacting a compound of formula (II): 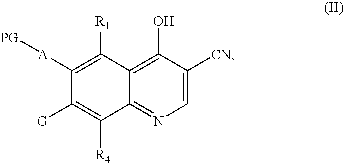
with a reagent of formula POX₃ in the presence of silica gel at a temperature greater than about 75° C.,

wherein X is halo,

PG is a protecting group selected from the group consisting of acyl, CH₃OC(O)—, EtOC(O)—, Fmoc, trifluoroacetamide, Troc, Phenoc, benzamide, Teoc and cyclic imides such as pthalimide, maleimide and pyrroles (e.g. 2,5-dimethylpyrrole);

A is O, NR, or S,

R is H, alkyl, alkenyl or alkynyl, and

G, R₁ and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 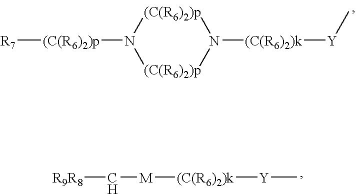
R₇—(C(R₆)₂)_{g}—Y—, R₇—(C(R₆)₂)ₚ-M-(C(R₆)₂)ₖ—Y—, or Het-(C(R₆)₂)_{q}W(C(R₆)₂—Y—,

or R₁ and R₄ are as defined above and G is R₂—NH—,

or if any of the substituents R₁, R₄ or G are located on contiguous carbon atoms then they may be taken together as the divalent radical —O—C(R₆)₂—O;

Y is a divalent radical selected from the group consisting of

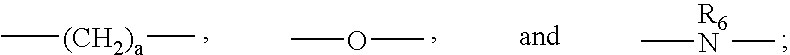

R₇ is —NR₆R₆, —OR₆, -J, —N(R₆)₃⁺, or —NR₆(OR₆),

M is >NR₆, —O—, >N—(C(R₆)₂)ₚNR₆R₆, or >N—(C(R₆)₂)ₚ—OR₆,

W is >NR6, —O— or is a bond;

Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and

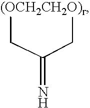

wherein Het is optionally mono- or di-substituted on carbon or nitrogen with R₆, optionally mono- or di-substituted on carbon with hydroxy, —N(R₆)₂, or —OR₆, optionally mono or di-substituted on carbon with the mono-valent radicals —(C(R₆)₂)ₛOR₆ or —(C(R₆)₂)ₛN(R₆)₂, and optionally mono or di-substituted on a saturated carbon with divalent radicals —O— or —O(C(R₆)₂)ₛO—;

R₆ is hydrogen, alkyl of 1-6 carbon atoms, alkenyl of 2-6 carbon atoms, alkynyl of 2-6 carbon atoms, cycloalkyl of 3-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom,

R₂ is selected from the group consisting of

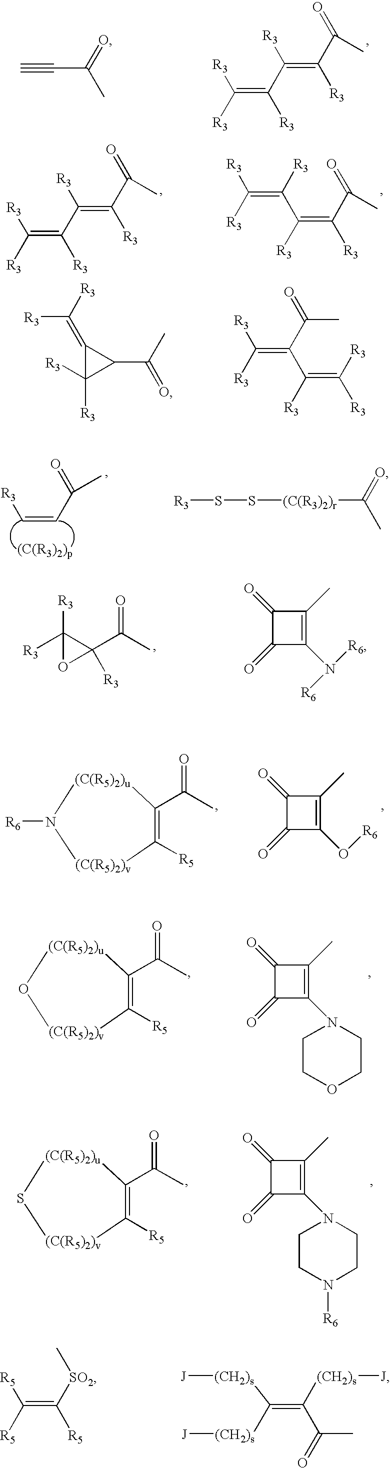

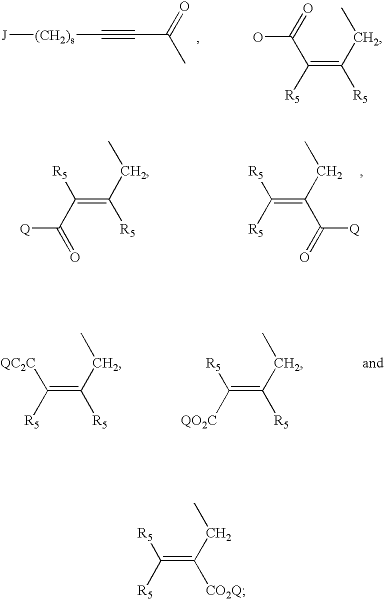

R₃ is independently hydrogen, alkyl of 1-6 carbon atoms, carboxy, carboalkoxy of 1-6 carbon atoms, phenyl, carboalkyl of 2-7 carbon atoms,

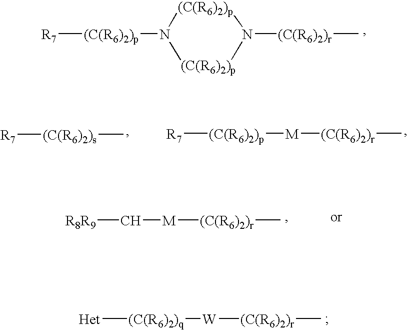

R₅ is independently hydrogen, alkyl of 1-6 carbon atoms, carboxy, carboalkoxy of 1-6 carbon atoms, phenyl carboalkyl of 2-7 carbon atoms,

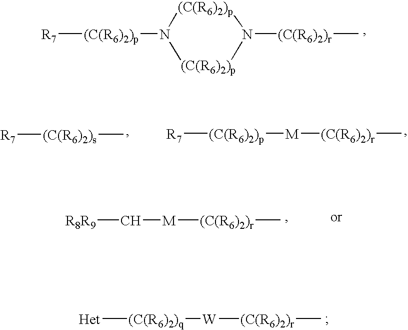

R₈ and R₉ are each independently —(C(R₆)₂)ᵣNR₆R₆, or —(C(R₆)₂)ᵣOR₆,

J is independently hydrogen, chlorine, fluorine, or bromine,

Q is an alkyl of 1-6 carbon atoms or hydrogen,

a is 0 or 1,

g is 1-6,

k is 0-4,

n is 0-1,

m is 0-3,

p is 2-4,

q is 0-4,

r is 1-4,

s is 1-6,

u is 0-4 and v is 0-4, wherein the sum of u+v is 2-4,

x is 0-3,

y is 0-1, and

z is 0-3;

or a salt thereof.

### DETAILED DESCRIPTION OF THE INVENTION

The method of the present invention for preparing 4-halogenated quinoline compounds has multiple distinct advantages over previous methods of preparing such intermediate compounds. Most significantly, it does not result in the formation of ball tar, which is an obstacle for stirring at the pilot plant scale. In addition, the present method generates the intermediate in significantly higher yields than the prior methods. In the prior methods, yields were typically in the range of 30 to 50%, whereas the method of the present invention provides yields greater than 50%, typically about 70% or greater. Furthermore, the current method reduces the reagent required to halogenate the starting compound. The amount of POX₃ employed in the present invention should be an amount effective to produce a yield of greater than 50%, and typically will be in a range of about 2.0 to about 5.0 equivalents. In the method of the present invention excellent yields may be obtained with only 2.0 equivalents of POX₃, whereas 2.5 to 5.0 equivalents was required using the prior art methods that resulted in lower yields. Thus, the present method is more cost efficient for large scale synthesis.

The quinoline compounds of the present invention have a protecting group (PG), selected from the group consisting of acyl, CH₃OC(O)—, EtOC(O)—, Fmoc, trifluoroacetamide, Troc, Phenoc, benzamide, Teoc and cyclic imides such as pthalimide, maleimide and 2,5-dimethylpyrrole, at substituent A attached to the 6-position of the quinoline ring system. The protecting groups are stable under the conditions of the present method, but can be subsequently removed so that the 6-position can be further modified later in the synthesis.

With these advantages, the present method overcomes many of the limitations of previous methods, resulting in higher throughput and a more cost-effective way to prepare quinoline core compounds for use in the manufacture of biologically active compounds, such as, RTK inhibitors.

For purposes of this invention, the term “alkyl” includes both straight and branched alkyl moieties, which can contain as many as 12 carbon atoms. Preferably, the alkyl moiety contains between 1 to 6 carbon atoms, though 1 to 4 carbon atoms is more preferable.

For purposes of this invention, the term “alkenyl” refers to a radical aliphatic hydrocarbon containing one double bond and includes both straight and branched alkenyl moieties of 2 to 6 carbon atoms. Such alkenyl moieties may exist in the E or Z configurations; the compounds of this invention include both configurations.

For purposes of this invention, the term “alkynyl” includes both straight chain and branched moieties containing 2 to 6 carbon atoms having at least one triple bond.

For purposes of this invention, the term “cycloalkyl” refers to alicyclic hydrocarbon groups having 3 to 12 carbon atoms and includes but is not limited to: cyclopropyl, cyclobutyl, cyclopentyl, cyclohexyl, cycloheptyl, norbornyl, or adamantyl.

For purposes of this invention, the term “aryl” is defined as an aromatic hydrocarbon moiety and may be substituted or unsubstituted. An aryl group preferably contains 6 to 12 carbon atoms and may be selected from, but not limited to, the group: phenyl, α-naphthyl, β-naphthyl, biphenyl, anthryl, tetrahydronaphthyl, phenanthryl, fluorenyl, indanyl, biphenylenyl, acenaphthenyl, acenaphthylenyl, or phenanthrenyl groups. An aryl group may be optionally mono-, di-, tri- or tetra-substituted with substituents selected from, but not limited to, the group consisting of alkyl, acyl, alkoxycarbonyl, alkoxy, alkoxyalkyl, alkoxyalkoxy, cyano, halogen, hydroxy, nitro, trifluoromethyl, trifluoromethoxy, trifluoropropyl, amino, alkylamino, dialkylamino, dialkylaminoalkyl, hydroxyalkyl, alkoxyalkyl, alkylthio, —SO₃H, —SO₂NH₂, —SO₂NHalkyl, —SO₂N(alkyl)₂, —CO₂H, CO₂NH₂, CO₂NHalkyl, and —CO₂N(alkyl)₂. Preferred substituents for aryl and heteroaryl include: alkyl, halogen, amino, alkylamino, dialkylamino, trifluoromethyl, trifluoromethoxy, arylalkyl, and alkylaryl.

For purposes of this invention, the term “heteroaryl” is defined as an aromatic heterocyclic ring system (monocyclic or bicyclic) where the heteroaryl moieties are five or six membered rings containing 1 to 4 heteroatoms selected from the group consisting of S, N, and O, and include but are not limited to: (1) furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, pyrrolidinyl; (2) a bicyclic aromatic heterocycle where a phenyl, pyridine, pyrimidine or pyridizine ring is: (i) fused to a 6-membered aromatic (unsaturated) heterocyclic ring having one nitrogen atom; (ii) fused to a 5 or 6-membered aromatic (unsaturated) heterocyclic ring having two nitrogen atoms; (iii) fused to a 5-membered aromatic (unsaturated) heterocyclic ring having one nitrogen atom together with either one oxygen or one sulfur atom; or (iv) fused to a 5-membered aromatic (unsaturated) heterocyclic ring having one heteroatom selected from O, N or S. Preferably a bicyclic heteroaryl group contains 8 to 12 carbon atoms.

For purposes of this invention, the term “alkoxy” is defined as C₁-C₆-alkyl-O—; wherein alkyl is as defined above.

For purposes of this invention, the term “alkanoyloxymethyl” is defined as —CH₂OC(O)R, wherein R is alkyl of 1 to 6 carbon atoms.

For purposes of this invention, the terms “alkylaminoalkoxy” and “dialkylaminoalkoxy” refer to alkylamino and dialkylamino moieties with one or two alkyl groups (the same or different) bonded to the nitrogen atom which is attached to an alkoxy group of 1 to 6 carbon atoms. Preferably a dialkylaminoalkoxy moiety consists of 3 to 10 carbon atoms and an alkylaminoalkoxy moiety consists of from 2 to 9 carbon atoms.

For purposes of this invention, the term “alkylthio” is defined as C₁-C₆-alkyl-S.

For purposes of this invention, “alkoxyalkyl” and “alkylthioalkyl” denote an alkyl group as defined above that is further substituted with an alkoxy or alkylthio as defined above. A preferred alkoxyalkyl moiety is alkoxymethyl (e.g. alkoxy-CH₂—).

For purposes of this invention, the term “hydroxy” is defined as a HO-moiety.

For purposes of this invention, the term “hydroxylalkyl” is defined as a HO-alkyl-moiety, wherein the alkyl moiety consists of 1 to 6 carbons.

For purposes of this invention, the term “benzoylamino” is defined as a Ph-OC(O)NH— moiety.

For purposes of this invention, the terms “monoalkylamino” and “dialkylamino” refer to moieties with one or two alkyl groups wherein the alkyl chain is 1 to 6 carbons and the groups may be the same or different.

For purposes of this invention, the terms “monoalkylaminoalkyl” and “dialkylaminoalkyl” refer to monoalkylamino and dialkylamino moieties with one or two alkyl groups (the same or different) bonded to the nitrogen atom which is attached to an alkyl group of 1 to 6 carbon atoms. Preferably a dialkylaminoalkyl moiety consists of 3 to 10 carbon atoms and an alkylaminoalkyl moiety consists of from 2 to 9 carbon atoms.

For purposes of this invention. the term “mercapto” is defined as a —SH moiety.

For purposes of this invention, the term “carboxy” is defined as a —COOH moiety.

For purposes of this invention, the term “alkenoylamino” and “alkynoylamino” are defined as a —NH—COOR moiety, wherein R is alkenyl or alkynyl of 3 to 8 carbon atoms.

For purposes of this invention, the term “carboalkoxy” is defined as —CO₂R, wherein R is alkyl of 1 to 6 carbon atoms.

For purposes of this invention, the term “carboalkyl” is defined as —COR, wherein R is alkyl of 1 to 6 carbon atoms.

For purposes of this invention, the term “carboxyalkyl” is defined as a HOOCR-moiety, wherein R is alkyl of 1 to 6 carbon atoms.

For purposes of this invention, the term “carboalkoxyalkyl” is defined as a —R—CO₂—R′ moiety, wherein R and R′ are alkyl and together consist of from 2 to 7 carbon atoms.

For purposes of this invention, the term “aminoalkyl” is defined as H₂N-alkyl, wherein the alkyl group consists of 1 to 5 carbon atoms.

For purposes of this invention, the term “azido” is defined as a radical of formula —N₃.

For purposes of this invention, the term “alkanoylamino” is defined as a —NH—COOR moiety, wherein R is alkyl of 1 to 6 carbon atoms.

For purposes of this invention, the term “acyl” is defined as a radical of formula —(C═O)-alkyl or —(C═O)-perfluoroalkyl, wherein the alkyl radical or perfluoroalkyl radical is 1 to 6 carbon atoms, i.e. C₂ to C₇ alkanoyl or C₂ to C₇ perfluoroalkanoyl; preferred examples include but are not limited to, acetyl, propionyl, butyryl, trifluoroacetyl. The trifluoroacetyl is preferably attached to —NR— so that the compound is a trifluoroacetamide.

For purposes of this invention, the term “alkylsulfinyl” is defined as a R′SO— radical, where R′ is an alkyl radical of 1 to 6 carbon atoms.

For purposes of this invention, “alkylsulfonyl” is defined as a R′SO₂— radical, where R′ is an alkyl radical of 1 to 6 carbon atoms.

For purposes of this invention, “alkylsulfonamido,” “alkenylsulfonamido,” “alkynylsulfonamido” are defined as R′ SO₂NH— radicals, where R′ is an alkyl radical of 1 to 6 carbon atoms, an alkenyl radical of 2 to 6 carbon atoms, or an alkynyl radical of 2 to 6 carbon atoms, respectively.

The term “substituent” is used herein to refer to an atom radical, a functional group radical or a moiety radical that replaces a hydrogen radical on a molecule. Unless expressly stated otherwise, it should be assumed that any of the substituents may be optionally substituted with one or more groups selected from: alkyl, halogen, haloalkyl, hydroxyalkyl, nitro, amino, hydroxy, cyano, alkylamino, dialkylamino, alkoxy, haloalkoxy, alkoxyalkyl, alkoxyalkoxy, oxo, alkylthio, mercapto, haloalkylthio, aryl, aryloxy, arylthio, heteroaryl, heteroaryloxy, heteroarylthio, acyl, —CO₂-alkyl, —SO₃H, —SO₂NH₂, —SO₂NH-alkyl, —SO₂NH-(alkyl)₂, —CO₂H, —CO₂NH₂, —CO₂NH-alkyl and —CO₂N-(alkyl)₂.

For purposes of this invention, a “halogen” or “halo” radical is one of the non-metallic elements found in group VII A of the periodic table. Accordingly, a halogen of the present invention is a monovalent moiety which is derived from fluorine, chlorine, bromine, iodine or astatine. Preferred halogens are selected from the group consisting of chloro, fluoro and bromo.

For the purposes of this invention, the term “substituted” refers to where a hydrogen radical on a molecule has been replaced by another atom radical, a functional group radical or a moiety radical; these radicals being generally referred to as “substituents.”

For the purposes of this invention, the term “yield” refers to an amount of compound produced by a reaction or process. Typically, this refers to the amount of a compound recovered after any purification steps have been taken, for example, after recrystallization or chromatography. This amount is usually expressed as a percentage of product recovered relative to the amount of starting material and is generally based upon the quantity of moles. For example, if 1.0 mole of starting material is reacted and the recovered product after purification, is 0.73 moles, then the product was prepared in a 73% yield. One skilled in the art would readily understand this concept.

For purposes of this invention, the term “protecting group” refers to a group introduced into a molecule to protect a sensitive functional group or specific position on the molecule from reacting when the molecule is exposed to reagents or conditions to transform or react another part of the molecule. Thereafter the protecting group can be removed. Suitable protecting groups are well known in the art and include acid-labile, base-labile, photoremovable, or removable under neutral conditions. See, e.g., Green, Protecting Groups in Organic Synthesis, Wiley, pp. 218-288 (1985), which is incorporated herein by reference.

For the present invention, suitable protecting groups are acyl, CH₃OC(O)—, EtOC(O)—, Fmoc, trifluoroacetamide, Troc, Phenoc, benzamide, Teoc and cyclic imides such as pthalimide, maleimide and 2,5-dimethylpyrrole. In one preferred embodiment, the protecting group is acyl, most preferably acetyl. Where the protecting group is trifluoroacetamide or benzamide, PG is N-trifluoroacetyl or N-benzoyl attached to the group —NR—. Where the protecting groups is a cyclic imide such as pthalimide, maleimide, or 2,5-dimethylpyrrole, the group PG-NR— attached to the 6-position of the quinoline ring system is the radical derived from the cyclic imide by removal of the hydrogen atom attached to the imide-nitrogen atom, for instance, pthalimido, maleimido or 2,5-dimethylpyrrol-1-yl.

The compounds prepared by the method of this invention may contain an asymmetric carbon atom and may thus give rise to stereoisomers, such as enantiomers and diastereomers. The stereioisomers of the instant invention are named according to the Cahn-Ingold-Prelog System. While shown without respect to stereochemistry in formula (I), the present invention includes all the individual possible stereoisomers; as well as the racemic mixtures and other mixtures of R and S stereoisomers (scalemic mixtures which are mixtures of unequal amounts of enantiomers) and salts thereof. It should be noted that stereoisomers having the same relative configuration at a chiral center may nevertheless have different R and S designations depending on the substitution at the indicated chiral center.

The foregoing method also includes the preparation and forming of salts of the compounds of formula (I). As a base, quinoline can form various acid salts. The salts of the compounds of formula (I) may be readily prepared by methods known to persons of ordinary skill in the art. For the purpose of this invention, salts are those derived from organic and inorganic acids. Such organic and inorganic acids may be acetic, lactic, citric, tartaric, succinic, maleic, malonic, gluconic, hydrochloric, hydrobromic, phosphoric, nitric, sulfuric, methanesulfonic, and similarly known acceptable acids. Common mineral acids are HCl, H₂SO₄ and HNO₃. These lists are intended only to provide examples and are not intended to be exhaustive. Thus, the present invention should not be viewed as limited to these examples.
General Synthesis

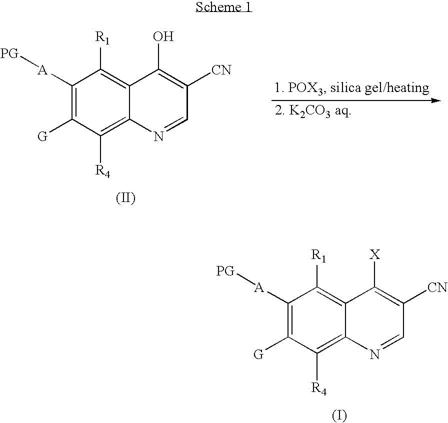

In Scheme 1, X, PG, A, G, R₁ and R₄ are as defined above.

The method depicted in Scheme 1 shows that a compound of formula (II) can be converted to a compound of formula (I) using a reagent of the formula POX₃ in the presence of silica gel. These quinoline intermediates can then be further substituted at the 4-position by reacting them with a nucleophilic reagent.

This reaction is generally heated to about 75° C. or greater, but preferably it is heated in the range of about 80° C. to about 85° C. For this reason acetonitrile is a preferred solvent, though one skilled in the art would know of other solvents appropriate for this reaction.

In a preferred embodiment, the phosphoryl halide used is phosphoryl chloride.

In another preferred embodiment, about 2.0 equivalents of silica gel are used in the reaction relative to the starting hydroxy compound.

In a preferred embodiment of the method of the present invention, A is NR, wherein R is H or alkyl.

In another embodiment of the method of the present invention, the method further comprises the steps of filtering the reaction mixture through diatomaceous earth, e.g celite, quenching the filtrate with a basic solution, and then filtering the quenched mixture to isolate the compound of formula (I). More preferably, the basic solution is K₂CO₃ dissolved in water.

This method provides the desired compound of formula (I) in yields greater than about 50%. Often the yields are greater than about 70%.

In another embodiment of the method of the present invention, the compounds prepared by this method are defined by G, R₁ and R₄ each independently being H, alkyl, alkoxy, CF₃O—, CF₃— and —CN. More preferable R₁ and/or R₄ are H, and G is alkoxy, particularly preferable is where G is ethoxy.

The following examples are set forth to aid in an understanding of the invention, and are not intended, and should not be construed, to limit in any way the invention set forth in the claims that follow thereafter.

### EXAMPLE 1

### Preparation of 4-chloro-3-cyano-7-ethoxy-6-acetylamino quinoline

3-cyano-7-ethoxy-4-hydroxy-6-acetylamino quinoline (150 g, 0.474 mol) was stirred with silica gel (60 g) in acetonitrile (1.35 L). The brown suspension was heated to 78-82° C. Phosphorus oxychloride (146 g, 0.949 mol) was added over 30-40 min. The mixture was stirred at 78-82° C. for 1-2 hrs then cooled to 40-45° C., filtered over a celite pad and washed with acetonitrile. The filtrates were quenched in a potassium carbonate solution (262 g, 1.9 mol) in water (1.8 L) at 0-5° C. over 45 min. The brownish suspension was stirred at 5-20° C. for at least 2 hours then filtered and washed with water. The brown/tan solid was dried in a vacuum oven at 50° C. to yield 105 g (76.5%).

HPLC

Strength 89.9%

Tot imp.=4.87%

Sing. imp.=1.28%

GC(CH₃CN)=0.83%

Water-content data determined on the basis of weight decrease in a loss on drying (LOD) test or determined by the Karl-Fisher method (KF)

KF=0.91%

LOD=1.3%.

The method of this invention can be used to prepare compounds disclosed in U.S. Pat. No. 6,002,008, which is incorporated in its entirety by reference. The conversion of compound of formula (I) to a compound of formula (III) below can be achieved by one skilled in the art by methods disclosed in U.S. Pat. No. 6,002,008. A method of preparing a compound of formula (III): 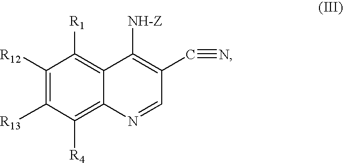
wherein:

Z is substituted phenyl;

R₁ is hydrogen;

R₄ is hydrogen;

R₁₂ and R₁₃ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, aminoalkyl of 1-4 carbon atoms, N-alkylaminoalkyl of 2-7 carbon atoms, N,N-dialkylaminoalkyl of 3-14 carbon atoms, phenylamino, benzylamino,

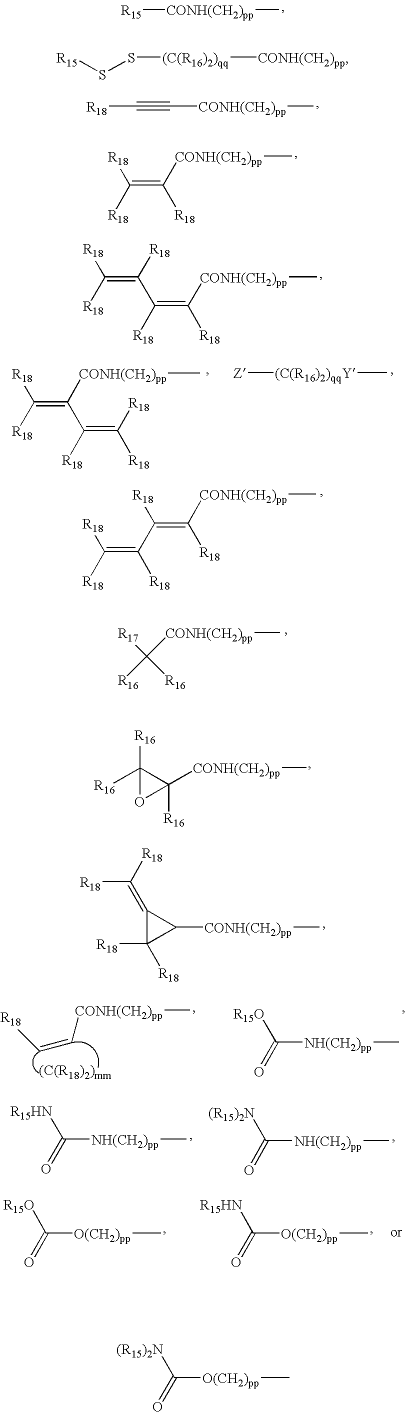

R₁₅ is alkyl of 1-6 carbon atoms, alkyl optionally substituted with one or more halogen atoms, phenyl, or phenyl optionally substituted with one or more halogen, alkoxy of 1-6 carbon atoms, trifluoromethyl, amino, nitro, cyano, or alkyl of 1-6 carbon atoms groups;

R₁₆ is hydrogen, alkyl of 1-6 carbon atoms, or alkenyl of 2-6 carbon atoms;

R₁₇ is chloro or bromo

R₁₈ is hydrogen, alkyl of 1-6 carbon atoms, aminoalkyl of 1-6 carbon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms, azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo;

Y′ is —NH—, —O—, —S—, or —NR—;

Z′ is amino, hydroxy, alkoxy of 1-6 carbon atoms, alkylamino wherein the alkyl moiety is of 1-6 carbon atoms, dialkylamino wherein each of the alkyl moieties is of 16 carbon atoms, morpholino, piperazino, N-alkylpiperazino wherein the alkyl moiety is of 1-6 carbon atoms, or pyrrolidino;

mm=1-4, qq=1-3, and pp=0-3;

any of the substituents R₁, R₁₂, R₁₃, or R₄ that are located on contiguous carbon atoms can together be the divalent radical —O—C(R₁₈)₂—O—;

or a pharmaceutically acceptable salt thereof with the proviso that R₁₂ is linked to the quinoline at the 6-position by an oxygen, sulfur or nitrogen atom;
comprising the step of reacting a compound of formula (II): 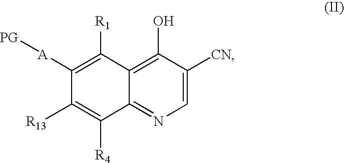
with a reagent of formula POX₃ in the presence of silica gel at a temperature greater than about 75° C.,
wherein:

X is halo;

PG is a protecting group selected from the group consisting of acyl, CH₃OC(O)—, EtOC(O)—, Fmoc, Troc, Phenoc, N-benzoyl, Teoc;

A is O, NR, or S;

R is H, alkyl, alkenyl, or alkynyl;

or the group PG-NR— is protected amino in the form of a radical derived from a cyclic imide by removal of the hydrogen atom attached to the imide-nitrogen atom; and

R₁, R₄ and R₁₃ are as defined above for formula (III)
to form the compound of formula (I): 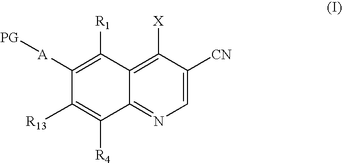
and converting the compound of formula (I) to the compound of formula (III).

A method of preparing (E)-N-{4-[3-chloro-4-(2-pyridinylmethoxy)anilino]-3-cyano-7-ethoxy-6-quinolinyl}-4-(dimethylamino)-2-butenamide or a pharmaceutically acceptable salt thereof; which comprises reacting 3-cyano-7-ethoxy-4-hydroxy-6-(protected amino)quinoline with a reagent of formula POX₃ (wherein X is halo) in the presence of silica gel at a temperature greater than about 75° C. to form 3-cyano-7-ethoxy-4-halo-6-(protected amino)quinoline and converting 3-cyano-7-ethoxy-4-halo-6-(protected amino)quinoline into (E)-N-{4-[3-chloro-4-(2-pyridinylmethoxy)anilino]-3-cyano-7-ethoxy-6-quinolinyl}-4-(dimethylamino)-2-butenamide or a pharmaceutically acceptable salt thereof.

What is claimed is:

1. A method of preparing a compound of formula (I): 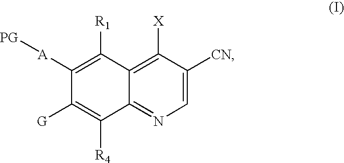
comprising the step of reacting a compound of formula (II): 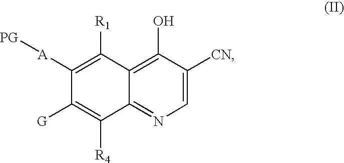
with a reagent of formula POX₃ in the presence of silica gel at a temperature greater than about 75° C.,
wherein:
X is halo;
PG is a protecting group selected from the group consisting of acyl, CH₃OC(O)—, EtOC(O)—, Fmoc, Troc, Phenoc, N-benzoyl, Teoc;
A is O, NR, or S;
R is H, alkyl, alkenyl, or alkynyl;
or the group PG-NR— is protected amino in the form of a radical derived from a cyclic imide by removal of the hydrogen atom attached to the imide-nitrogen atom;
and
G, R₁ and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 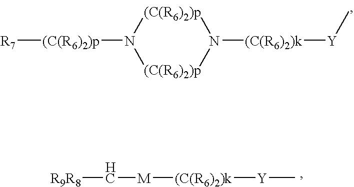
R₇—(C(R₆)₂)_{g}—Y—, R₇—(C(R₆)₂)ₚ-M-(C(R₆)₂)ₖ—Y—, or Het-(C(R₆)₂)_{q}W(C(R₆)₂—Y—;
or R₁ and R₄ are as defined above and G is R₂—NH—;
or R₄ and G may be taken together as the divalent radical —O—C(R₆)₂—O;
Y is a divalent radical selected from the group consisting of 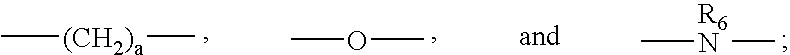
R₇ is —NR₆R₆, —OR₆, -J, —N(R₆)₃⁺, or —NR6(OR6);
M is >NR₆, —O—, >N—(C(R₆)₂)ₚNR₆R₆, or >N—(C(R₆)₂)ₚ—OR₆;
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 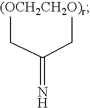
wherein Het is optionally mono- or di-substituted on carbon or nitrogen with R₆, optionally mono- or di-substituted on carbon with hydroxy, —N(R₆)₂, or —OR₆, optionally mono or di-substituted on carbon with the mono-valent radicals —(C(R₆)₂)ₛOR₆ or —(C(R₆)₂)ₛN(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 3-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂, is selected from the group consisting of 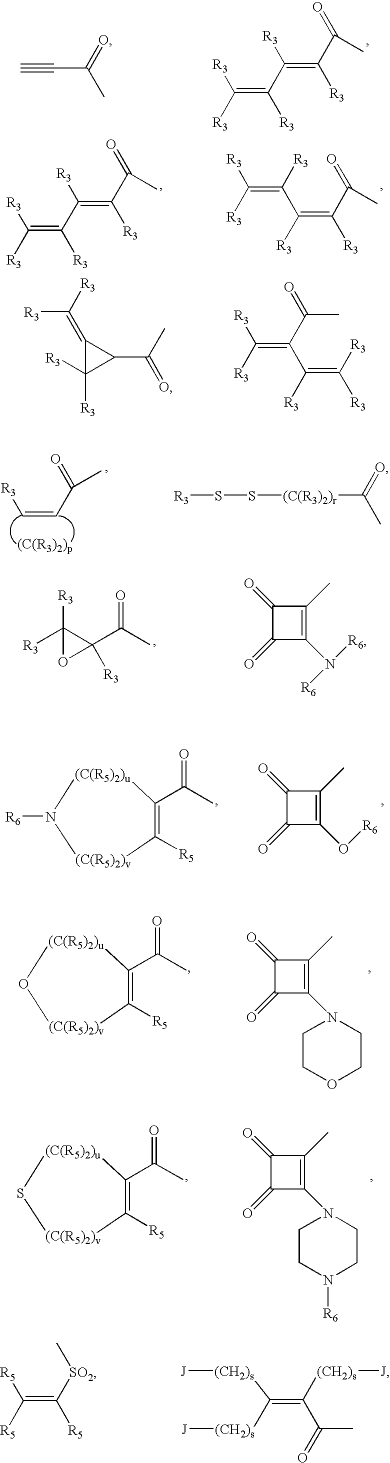 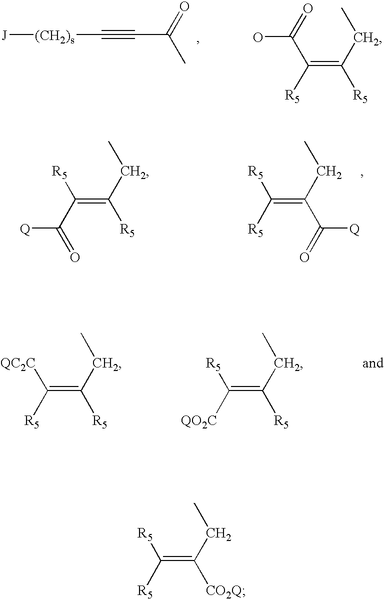
R₃ is independently hydrogen, alkyl of 1-6 carbon atoms, carboxy, carboalkoxy of 1-6 carbon atoms, phenyl, carboalkyl of 2-7 carbon atoms, 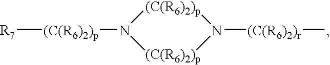
R₇—(C(R₆)₂)ₛ—,
R₇—(C(R₆)₂)ₚ-M-(C(R₆)₂)ᵣ—, R₈R₉—CH—
M-(C(R₆)₂)ᵣ—, or
Het-(C(R₆)₂)_{q}—W—(C(R₆)₂)ᵣ—;
R₅ is independently hydrogen, alkyl of 1-6 carbon atoms, carboxy, carboalkoxy of 1-6 carbon atoms, phenyl carboalkyl of 2-7 carbon atoms, 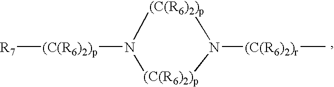
R₇—(C(R₆)₂)ₛ—,
R₇—(C(R₆)₂)ₚ-M-(C(R₆)₂)ᵣ—, R₈R₉—CH—
M-(C(R₆)₂)ᵣ—, or
Het-(C(R₆)₂)_{q}—W—(C(R₆)₂)ᵣ—;
R₈, and R₉ are each independently —(C(R₆)₂)ᵣNR₆R₆, or —(C(R₆)₂)ᵣ OR₆;
J is independently hydrogen, chlorine, fluorine, or bromine;
Q is an alkyl of 1-6 carbon atoms or hydrogen;
a=0 or 1;
g=1-6;
k=0-4;
n is 0-1;
m is 0-3;
p=2-4;
q=0-4;
r=1-4;
s=1-6;
u=0-4 and v=0-4, wherein the sum of u+v is 2-4;
x=0-3;
y=0-1;
z=0-3;
or a salt thereof wherein “acyl” in the definition of PG is defined as C₂ to C₇ alkanoyl or C₂ to C₇ perfluoroalkanoyl.

2. The method of claim 1, wherein A is NR and R is H or alkyl.

3. The method of claim 1, wherein X is Cl.

4. The method of claim 1, wherein the temperature is between about 80° C. and 85° C.

5. The method of claim 1, wherein PG is acetyl.

6. The method of claim 1, wherein G, R₁ and R₄ are each independently H, alkyl, alkoxy, trifluoromethyl, trifluoromethoxy and CN.

7. The method of claim 6, wherein R₁ is H.

8. The method of claim 7, wherein R₄ is H.

9. The method of claim 8, wherein G is alkoxy.

10. The method of claim 9, wherein G is ethoxy.

11. The method of claim 1, further comprising the steps of:
1. filtering the reaction mixture through diatomaceous earth;
2. quenching the filtrate with a basic solution; and
3. filtering the quenched mixture to isolate the compound of formula (I).

12. The method of claim 11, wherein the basic solution is K₂CO₃ in water.

13. The method of claim 1, wherein the compound of formula (I) is yielded in greater than about 50%.

14. The method of claim 1, wherein the compound of formula (I) is yielded in greater than about 70%.

15. The method of claim 1, wherein about 2.0 equivalents of silica gel are used in the reaction relative to the compound of formula (II).

16. The method of claim 1, wherein about 2.0 equivalents of POX₃ are used in the reaction relative to the compound of formula (II).

17. A method of preparing a compound of formula (I): 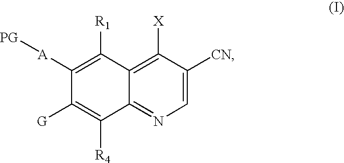
comprising the step of reacting a compound of formula (II): 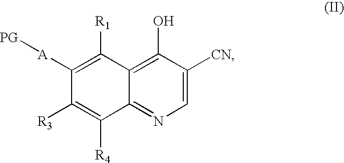
with a reagent of formula POX₃ in the presence of silica gel at a temperature greater than about 75° C.,
wherein:
X is halo;
PG-A- is 2,4-dimethylpyrrol-1-yl; and
G, R₁ and R₄ are as defined in claim 1.

18. A method of preparing a compound of formula (III): 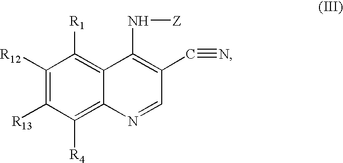
wherein:
Z is substituted phenyl;
R₁ is hydrogen;
R₄ is hydrogen;
R₁₂ and R₁₃ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, aminoalkyl of 1-4 carbon atoms, N-alkylaminoalkyl of 2-7 carbon atoms, N,N-dialkylaminoalkyl of 3-14 carbon atoms, phenylamino, benzylamino, 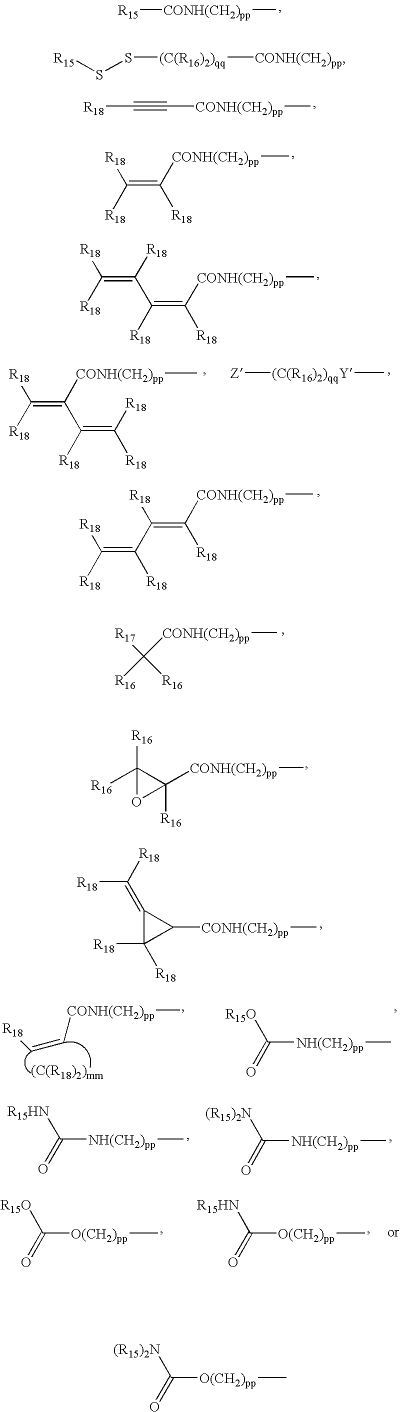
R₁₅ is alkyl of 1-6 carbon atoms, alkyl optionally substituted with one or more halogen atoms, phenyl, or phenyl optionally substituted with one or more halogen, alkoxy of 1-6 carbon atoms, trifluoromethyl, amino, nitro, cyano, or alkyl of 1-6 carbon atoms groups;
R₁₆ is hydrogen, alkyl of 1-6 carbon atoms, or alkenyl of 2-6 carbon atoms;
R₁₇ is chloro or bromo
R₁₈ is hydrogen, alkyl of 1-6 carbon atoms, aminoalkyl of 1-6 carbon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms, azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo;
Y′ is —NH—, —O—, —S—, or —NR—;
Z′ is amino, hydroxy, alkoxy of 1-6 carbon atoms, alkylamino wherein the alkyl moiety is of 1-6 carbon atoms, dialkylamino wherein each of the alkyl moieties is of 1-6 carbon atoms, morpholino, piperazino, N-alkylpiperazino wherein the alkyl moiety is of 1-6 carbon atoms, or pyrrolidino;
mm=1-4, qq=1-3, and pp=0-3;
any of the substituents R₁, R₁₂, R₁₃, or R₄ that are located on contiguous carbon atoms can together be the divalent radical —O—C(R₁₈)₂—O—;
or a pharmaceutically acceptable salt thereof with the proviso that R₁₂ is linked to the quinoline at the 6-position by an oxygen, sulfur or nitrogen atom;
comprising the step of reacting a compound of formula (II): 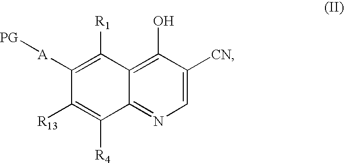
with a reagent of formula POX₃ in the presence of silica gel at a temperature greater than about 75° C.,
wherein:
X is halo;
PG is a protecting group selected from the group consisting of acyl, CH₃OC(O)—, EtOC(O)—, Fmoc, Troc, Phenoc, N-benzoyl, Teoc;
A is O, NR, or S;
R is H, alkyl, alkenyl, or alkynyl;
or the group PG-NR— is protected amino in the form of a radical derived from a cyclic imide by removal of the hydrogen atom attached to the imide-nitrogen atom; and
and
R₁, R₄ and R₁₃ are as defined above for formula (III) to form the compound of formula (I): 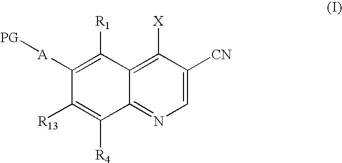
and converting the compound of formula (I) to the compound of formula (III).

19. A method of preparing (E)-N-{4-[3-chloro-4-(2-pyridinylmethoxy)anilino]-3-cyano-7-ethoxy-6-quinolinyl}-4-(dimethylamino)-2-butenamide or a pharmaceutically acceptable salt thereof; which comprises reacting 3-cyano-7-ethoxy-4-hydroxy-6-(protected amino)quinoline with a reagent of formula POX₃ (wherein X is halo) in the presence of silica gel at a temperature greater than about 75° C. to form 3-cyano-7-ethoxy-4-halo-6-(protected amino)quinoline and converting 3-cyano-7-ethoxy-4-halo-6-(protected amino)quinoline into (E)-N-{4-[3-chloro-4-(2-pyridinylmethoxy)anilino]-3-cyano-7-ethoxy-6-quinolinyl}-4-(dimethylamino)-2-butenamide or a pharmaceutically acceptable salt thereof.
